(19) United States
(12) Patent Application Publication
(10) Pub. No.: US 2019/0298963 A1
(43) Pub. Date: Oct. 3, 2019

(54) Mammalian Respiration Heater and Method

(71) Applicant: O & R Lifesaving Technology, L.L.C., Moses Lake, WA (US)

(72) Inventor: Richard Rincoe, Moses Lake, WA (US)

(73) Assignee: O & R Lifesaving Technology, L.L.C., Moses Lake, WA (US)

(21) Appl. No.: 15/939,003

(22) Filed: Mar. 28, 2018

(51) Int. Cl. A61M 16/16; A61M 16/08

(52) U.S. Cl. CPC A61M 16/16; A61M 16/142; A61M 2205/3368; A61M 16/08

(57) ABSTRACT

A mammalian respiration heater is provided including a container, a tube, and an aperture. The container has at least one wall configured to form a reservoir for containing liquid having a thermal capacity capable of rendering sensible heat to a user through respiration. The tube is carried for passage through the container in sealed relation at a first end and is configured to couple with a user's input respiration and a second end configured to draw in ambient air, and having a thermally conductive portion exposed in thermally conductive relation with the reservoir within the container. The aperture is provided in the container configured to enable admittance of liquid having an elevated temperature within the container. A method is also provided.

### TECHNICAL FIELD

This disclosure pertains to containers for carrying liquids. More particularly, this disclosure relates to apparatus and methods for carrying liquids and also providing hypothermia therapy through a respiratory system.

### BACKGROUND

Techniques are known for providing therapy when a body core temperature drops, such as by providing ingestion of heated liquids. However, ingestion of heated liquids within the gastrointestinal tract is limited due to capacity and does not provide a short path for the heat to pass to the cardiovascular system. Therefore, there exists a need to deliver heat more directly into a user's vascular system so that core body temperature can be elevated to treat and mitigate patient hypothermia under emergency and rescue situations, particularly in remote locations. There is a further need to provide such a solution while minimizing the need for additional equipment and weight that might need to be carried by a user over long distances in remote locations.

### SUMMARY

A fluid container, or canteen is provided configured to receive a quantity, or charge of heated fluid, such as water, and a user inhales through a tube extending through the container, transferring heat from the water through the tube via air into a user's bronchial tubes and lungs. The container is multi-use because it also serves as a canteen, or water bottle, and as a hot water bottle, thereby minimizing the amount of equipment needed to be carried for multiple functions.

According to one aspect, a mammalian respiration heater is provided including a container, a tube, and an aperture. The container has at least one wall configured to form a reservoir for containing liquid having a thermal capacity capable of rendering sensible heat to a user through respiration. The tube is carried for passage through the container in sealed relation at a first end and is configured to couple with a user's input respiration and a second end is configured to draw in ambient air, and having a thermally conductive portion exposed in thermally conductive relation with the reservoir within the container. The aperture is provided in the container configured to enable admittance of liquid having an elevated temperature within the container.

According to another aspect, a respiratory heater is provided having a container, a tube, and an opening. The container has a liquid reservoir. The tube is carried for passage through the liquid reservoir having a first open end and an opposed second open end each communicating with an exterior of the container. The opening is provided in the container configured to enable admittance of liquid with an elevated temperature into the container.

According to yet another aspect, a method is provided for mitigating hypothermia in a mammal. The method includes: providing a container having a tubular heat transfer tube extending through the container with opposed open ends exterior of the container; admitting elevated temperature fluid into the container about the tube; and drawing air at the first end of the tube from the second end of the tube drawn through the tube to elevate in temperature through conduction of the tube with the fluid and into a user's lungs.

### BRIEF DESCRIPTION OF THE DRAWINGS

FIG. 1 is a perspective view illustrating one version of a combination canteen and mammalian respiration heater according to one embodiment.

FIG. 2 is an exploded perspective view from above of one version of a canteen and mammalian respiration heater illustrating construction and molding of the canteen and heater according to one embodiment.

FIG. 3 is a simplified side view of the canteen and heater of FIG. 3 with a front portion of the canteen removed in cut-away view to show the heat exchanger tube.

FIG. 4 is a simplified perspective view of the canteen and heater of FIGS. 2 and 3 with a carrying pouch.

### DETAILED DESCRIPTION OF THE ILLUSTRATED EMBODIMENTS

This disclosure is submitted in furtherance of the constitutional purposes of the U.S. Patent Laws “to promote the progress of science and useful arts” (Article 1, Section 8).

FIG. 1 illustrates a canteen and mammalian respiration heater 10 according to one version suitable for use in remote, rugged, and adverse cold weather locations to implement remediation of hypothermia on a user. Canteen 10 includes a container, or vessel 12 comprising at least one wall portion having a concavity configured to contain fluid via coaction with gravity, or to completely encase such fluid and contain such fluid within a fluid sealed vessel. Such concavity provides a chamber inside of vessel 12 for containing fluid, such as water.

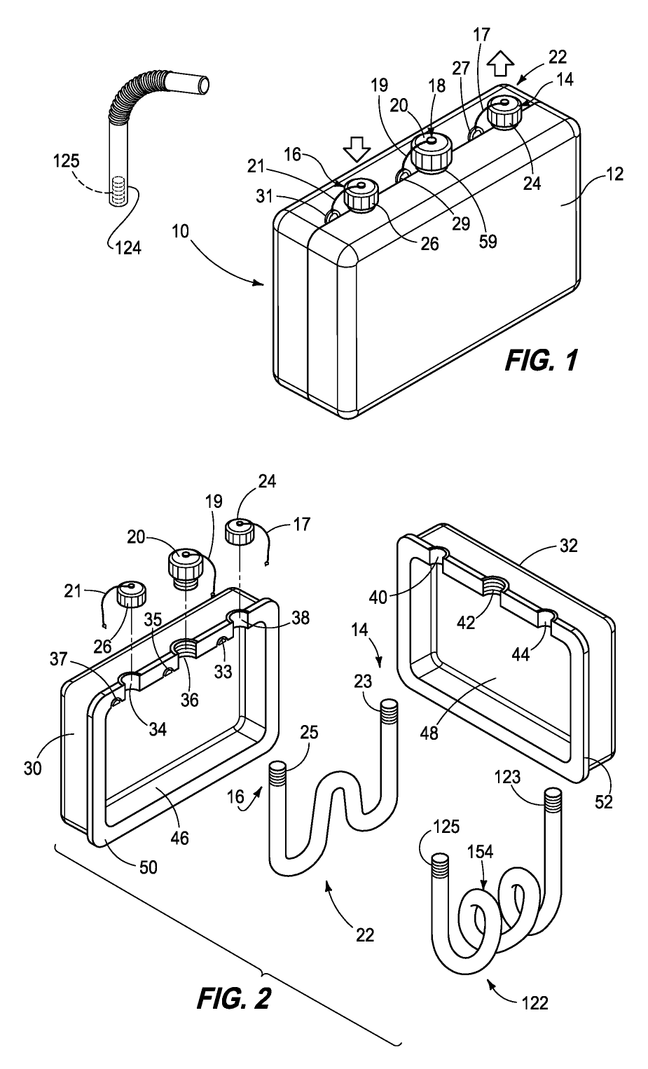

As shown in FIG. 1, container 12 is a generally rectangular-shaped vessel having six sidewalls contiguously formed with radiused, or rounded corners from plastic in a rotary molding process shown below with reference to FIG. 2. Optionally, container 12 can be constructed from metal, such as aluminum, or any other suitable material, such as composite materials. A heat exchanger tube 22 formed with a circuitous geometry (see FIG. 2) to increase length and heat exchange surface area is molded into vessel 12 during construction. Tube 22 facilitates use of canteen 10 as a hypothermia breathing system by a user under emergency situations to realize remedial hypothermia therapy to elevate body core temperature. A first open-end portion 14 provides a user inhalation inlet and an opposed second open-end portion 16 provides an ambient air intake inlet. However, it is understood in use that portion 14 can serve as portion 16, and vice versa.

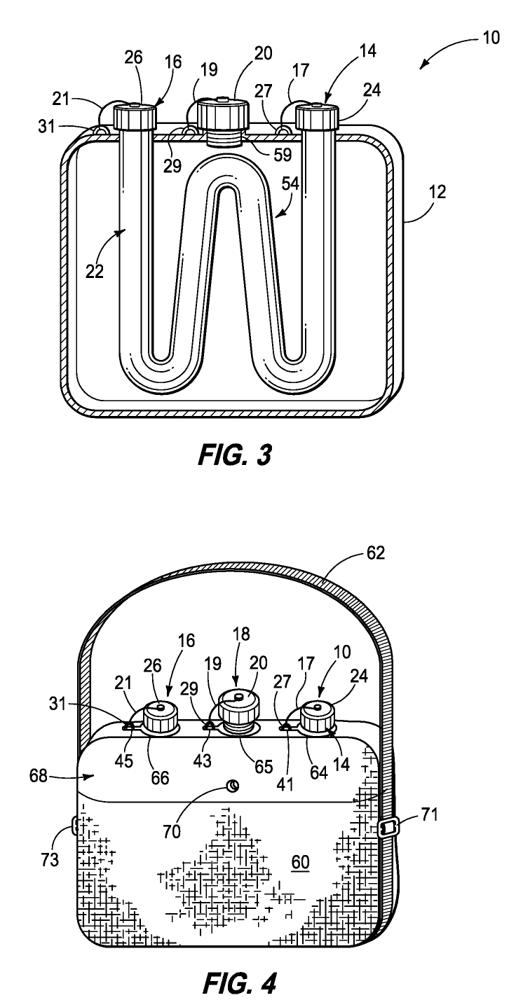

A closure assembly 18 is provided at the top of vessel 12 of FIG. 1 having a removable cap 20 and an inlet port, or opening 59 (see FIG. 3) comprising a tubular threaded end portion. Cap 20 includes a complementary threaded portion and a circumferential seal that engages the inlet port in threaded assembly. Cap 20 includes an attached chain lanyard 19 that affixes to an integrally molded eyelet 29 to prevent loss. According to one construction, a stud is provided atop cap 20 and an end ring is provided on chain lanyard that is trapped over the stud and configured to rotate about the stud when screwing and unscrewing the cap from the vessel 12.

End portions 14 and 16 of FIG. 1 each have a similar closure assembly comprising removable threaded caps 24 and 26 respectively affixed over threaded end portions 23 and 25 (see FIG. 2) of tube 22. Caps 24 and 26 each affix via respective dedicated chain lanyards 17 and 21 to eyelets 27 and 31 in a manner similar to cap 20 and eyelet 29. In one case, end portion 14 and 16 are each threaded male tube end segments 23 and 25. In another optional case, end portion 14 can receive in threaded complementary engagement a long flexible plastic breathing tube 124 having a complementarily female threaded internal portion 125 sized for threaded attachment onto end portion 14, such as used on a medical ventilator. Even further optionally, end portion 16 can be constructed to receive a tube similar to tube 124.

In use, an individual under risk of hypothermia fills vessel 12 of canteen 10 via closure assembly 18 with warm or hot fluid, such as water heated using a camp stove. The individual then affixes their mouth onto end portion 14 and draws ambient outside air from end portion 16 through heat exchanger tube 22 and into the individuals' lungs and bronchial passages where resultant heated air helps warm the individual from within their respiratory system and into their blood vessels. Preferably, such individual then exhales the air through their nose, or demates their mouth from end portion 14 and directly exhales to atmosphere so as to prevent reentry of the exhaled air into tube 22. Canteen 10, filled with heated water, can also be placed inside of a sleeping bag with an individual to further provide a thermal heat source to the user.

FIG. 2 illustrates in simplified exploded perspective view a molding process for creating one version of canteen 10 using rotary molding with polyethylene plastic beads. A pair of mating complementary mold members 30 and 32 are configured with complementary rectangular inner surface cavities 46 and 48, when clamped together, to form an outer surface of vessel 12. Optionally, canteen can be formed from aluminum and mold members 30 and 32 can be female stamping dies, with the two formed segments being welded, brazed, or soldered together along complementary edge seams while also welding, brazing or soldering in tube 22. Heat exchanger tube 22, comprising a copper tube of cylindrical cross section is inserted between mold members 30 and 32 in assembly. Optionally, tube 22 can be constructed of any other suitable structural material, such as aluminum, steel, plastic, or composite material. More particularly, end portion 14 is secured between a pair of complementarily sized semi-cylindrical edge gaps 38 and 40 in mold members 30 and 32, respectively. Similarly, end portion 16 is secured between a pair of complementarily sized semi-cylindrical edge gaps 34 and 44 in mold members 30 and 32, respectively. A pair of semi-cylindrical edge gaps 36 and 42 cooperate to provide cylindrical mold opening for inserting polyethylene beads into the resulting mold during a heating and rotary molding operation to form canteen 10. Edge gaps 36 and 42 also include female thread portions that serve to form a cylindrical array of integral threads on inlet port 59 (see FIG. 3). Finally, eyelet grooves 33, 35, and 37 are formed into mold member 30 in order to generate eyelets 27, 29, and 31, respectively during a molding operation. In the case of an aluminum or metal construction, such eyelets can be brazed onto the resulting canteen.

During such rotary molding operation shown in FIG. 2, plastic beads are introduced within such mold and the mold is rotated while placed in a heated environment, which causes the beads to melt and coat surfaces 46 and 48. Such action is carried out in a manner that minimizes coating of outer surfaces of tube 22 with melted plastic. The mold is then cooled, while still heating tube 22, and a resulting vessel is formed that encases and seals with tube 22, integrally forming tube 22 within such vessel, while minimizing coating of plastic on an outer surface of tube 22.

Optionally, vessel 12 of FIG. 1 can be made by separately molding two plastic half shells, inserting tube 22, and electro-sonically welding together the two half shells along a common border to form a vessel that seals and entraps tube 22. Further optional constructions include making vessel 12 from any suitable structural material, such as metals, plastics and glass, composite materials, or even using flexible membranes or layered fabrics, such as nylon and BPA-free plastic liners, or leather, or any other suitable flexible and water impervious membrane material. In such optional cases, tube 22 is sealed with the membrane via grommets or enforcement pass-through structures that seals tube 22 circumferentially with the membrane.

Also shown in FIG. 2, after separation of mold members 30 and 32, the vessel (not shown) is removed and threaded plastic end caps 24 and 26 are affixed over the ends of tube 22 and threaded plastic cap 20 is affixed over the inlet port. Lanyards 17, 19, and 21 are also affixed between the respective caps 24, 20, and 26 and eyelets 27, 29, and 31. As shown, cap 20 is sized larger than caps 24 and 26 at least in part to prevent inadvertent misplacement of a cap on a wrong threaded end portion 23, 25 (see FIG. 2), and 59 (see FIG. 3).

Optionally, heat exchanger tube 22 can take on any of a number of forms that provide sufficient surface area along with heat transfer from heated fluid in vessel 12 (of FIG. 1) into air that is being drawn through tube 22 by a user during inhalation and respiration. For example, heat exchanger tube 122 provides another configuration for a circuitous, or serpentine tube in a helical configuration having helical coils 154 formed integrally within tube 122 to increase surface area and distance within the vessel with threaded end portions 123 and 125. Further optionally, a straight tube, curved tube, or any other form of tube shape can be used. Even further optionally, further thermally conductive radiating surfaces, or fins can be provided on the inner surface and/or the outer surface of such tube. Yet even further optionally, heat exchanger tubes 22 and 122 can each be constructed from any suitable metal, plastic, composite, or other form of thermally conductive structural material capable of holding a tubular shape during inhalation, or negative pressure inspiration by a user.

FIG. 3 illustrates arrangement of heat exchanger tube 22 in cut away front view within canteen, or water bottle 10 with circuitous segment, or undulations 54 providing an increased length of tube 22 within vessel 12. Such increased length provides an increased surface area presented in thermally conductive relation with heated liquid that has been placed within vessel 12. Plastic end caps 24 and 26 are threaded coaxially atop open-end portions 14 and 16, and threaded plastic cap 20 is fitted coaxially atop threaded cylindrical inlet 59. In use, a user unscrews cap 20 from atop threaded inlet 59 and pours warm or hot water inside inner cavity of bottle 10. Cap 20 is then secured in sealing relation atop inlet 59. Caps 24 and 26 are then removed. A user then places their lips about threaded end portion 14 (see FIG. 2) and inhales, or attaches a longer version of cap 24 comprising corrugated inlet end tube 124 (see FIG. 1) and places their lips about an end of such tube and inhales. The user draws in ambient cold air via end portion 16 which serves as an inlet where it travels through tube 22 and is warmed before entering a user's bronchial tubes and lungs.

FIG. 4 illustrates canteen 10 placed within a cloth carrying pouch 60 that includes an adjustable length carrying strap 62 that affixes circumferentially about an outer periphery of pouch 60. A pair of metal slides 71 and 73, of stamped steel, are affixed at opposed ends of pouch 60 along a center slide bar with stitches of thread and a webbing tab (not shown) and serve to hold and guide strap 62 along pouch 60. End caps 24 and 26, affixed atop open end portions 14 and 16, respectively, extend through apertures 64 and 66 in pouch 60. Likewise, cap 20 extends through aperture 65 in pouch 60. Eyelets 27, 29 and 31 each extend through respective small slits 41, 43, and 45 that are contiguous with cylindrical apertures 64, 65, and 66 in pouch 60. Each of the slits is contiguous with respective small apertures 64, 65, and 66 to facilitate removal of canteen 10 from pouch 60. A cloth hinged cover assembly 68 enables loading of canteen 10 within pouch 60 and a snap fastener 70 secures cover assembly 68 in place to retain canteen 10 therein. Cover assembly 68 includes a sewn hinge (not shown) along an opposed back edge of pouch 60, opposite snap fastener 70.

As shown, cloth carrying pouch 60 comprises a duck canvas material with a cotton liner having a thickness sufficient to provide an insulation layer for warm/hot water that has been stored inside of canteen 10. The canteen, loaded in insulating pouch 60, can be worn inside of a user's jacket or within a sleeping bag, thereby providing a source of heat for the user when they are exposed to inclement cold weather. In addition, the cotton liner and canvas duck material of pouch 60 is hydrophilic, tending to absorb water. A user can dip pouch 60 into water when the ambient temperature is hot, and evaporative cooling will occur from the water absorbed into the fabric of pouch 60, causing cooling of canteen 10 and any liquid contents contained therein.

In use, a user that is in a location remote from a source of warm/hot water, can fill canteen 10 with a supply of water having a temperature elevated above surrounding, ambient air temperatures in order to use canteen 10 as a thermal source to mitigate hypothermia of lowered body temperatures. One such source can be created using a fire or a portable heat source, such as a camp stove, and a coffee or tea pot to heat up a supply of water. A funnel, or pouring spout/lip on a pot can be used to pour heated water into the canteen. Preferably, the heated water should be hot, but not boiling. After a user has performed a respiration heating operation with the filled canteen, the canteen can provide even further heating to a user by placing the canteen in a user's sleeping bag to experience a warm night's sleep. Effectively, the canteen becomes a hot water bottle in addition to being a source of hot/heated air that warms a body by heating the blood in a user's lungs that then circulates through their organs and extremities.

In mountainous areas, such as the Rocky Mountains, and in deserts it is not unusual to have very hot days followed by very cold nights. In such environments, the hypothermia breathing system provided by use of this canteen will undoubtedly serve both needs of keeping a user warm at night and preventing dehydration on a hot day. Such canteen can also benefit when a user carries extra water and a water filtration system.

Numerous exemplary alternative constructions can be realized for the above-described canteen. In one case, the canteen is a 2-quart capacity canteen configured for use when hiking, resting, or sleeping. Optionally, other sizes and shapes are suitable. A user can realize a need to use such canteen to mitigate hypothermia by monitoring whether their hands or feet feel cold which is an indication that the user's core temperature is losing heat. If a user feels very cold, it is a sign that they are in the early stages of hypothermia and they need to use the canteen, or they need to find a warm place to rest or recover. With a single device, a user can render the canteen usable to warm hands, feet and body core temperature by breathing warm air and raising body core temperature.

In compliance with the statute, the subject matter disclosed herein has been described in language more or less specific as to structural and methodical features. It is to be understood, however, that the claims are not limited to the specific features shown and described, since the means herein disclosed comprise example embodiments. The claims are thus to be afforded full scope as literally worded, and to be appropriately interpreted in accordance with the doctrine of equivalents.

I claim:

1. A mammalian respiration heater, comprising:
a container having at least one wall configured to form a reservoir for containing liquid having a thermal capacity capable of rendering sensible heat to a user through respiration;
a tube carried for passage through the container in sealed relation at a first end configured to couple with a user's input respiration and a second end configured to draw in ambient air, and having a thermally conductive portion exposed in thermally conductive relation with the reservoir within the container; and
an aperture provided in the container configured to enable admittance of liquid having an elevated temperature within the container.

2. The mammalian respiration heater of claim 1, wherein the container is a canteen.

3. The mammalian respiration heater of claim 2, wherein the canteen is a rotary molded plastic canteen.

4. The mammalian respirator heater of claim 1, wherein the tube comprises a tubular winding providing at least in part the thermally conductive portion.

5. The mammalian respirator heater of claim 1, wherein the thermally conductive portion comprises an undulate portion.

6. The mammalian respirator heater of claim 1, wherein the tube includes a circuitous portion.

7. The mammalian respirator heater of claim 7, wherein the circuitous portion is a tubular winding of thermally conductive coils.

8. The mammalian respirator heater of claim 1, wherein the tube comprises an aluminum tube.

9. The mammalian respirator heater of claim 1, wherein the container is a plastic container and the tube is a thermally conductive tube molded within the plastic container.

10. A respiratory heater, comprising:
a container having a liquid reservoir;
a tube carried for passage through the liquid reservoir having a first open end and an opposed second open end each communicating with an exterior of the container; and
an opening provided in the container configured to enable admittance of liquid with an elevated temperature into the container.

11. The respiratory heater of claim 10, wherein the container is a canteen having a closure assembly with a removable cap and inlet.

12. The respiratory heater of claim 11, wherein the tube comprises a thermally conductive heat transfer tube configured in sealed relation at a first end to couple with a user's input respiration and a second end to draw in ambient air, and having a thermally conductive portion exposed in thermally conductive relation with the reservoir within the container.

13. The respiratory heater of claim 10, wherein the tube comprises a circuitous segment of thermally conductive tubing.

14. The respiratory heater of claim 13, wherein the tube is an undulate tube of cylindrical cross section.

15. A method for mitigating hypothermia in a mammal, comprising:
providing a container having a tubular heat transfer tube extending through the container with opposed open ends exterior of the container;
admitting elevated temperature fluid into the container about the tube;
inspiring air at the first end of the tube from the second end of the tube drawn through the tube to elevate in temperature through conduction of the tube with the fluid and into a user's lungs.

16. The method of claim 15, wherein inspiring air comprises inhaling at the first end of the tube to draw air through the tube in thermally conductive relation via the tube with the elevated temperature fluid wherein drawing air comprises applying negative pressure (vacuum) at the first end of the tube to draw ambient air through the tube.

17. The method of claim 15, further comprising, after inspiring elevated temperature air into the user's lungs and bronchial tubes, holding the elevated temperature air in the user's lungs and bronchial tubes.

18. The method of claim 17, wherein holding the elevated temperature air realizes hypothermia remedial therapy.

19. The method of claim 15, wherein, after holding, further comprising exhaling the held air to atmosphere spaced from both ends of the tube.

20. The method of claim 15, further comprising placing the container in thermally conductive proximity with a user to warm at least a portion of the user.
